(19) United States
(12) Patent Application Publication
(10) Pub. No.: US 2020/0405982 A1
(43) Pub. Date: Dec. 31, 2020

(54) Aerosolization Conduit For Electronic Drug-Delivery Systems

(71) Applicant: Zayd A. Turbi, Irvine, CA (US)

(72) Inventor: Zayd A. Turbi, Irvine, CA (US)

(21) Appl. No.: 16/915,241

(22) Filed: Jun. 29, 2020

Related U.S. Application Data
(60) Provisional application No. 62/867,435, filed on Jun. 27, 2019.

(51) Int. Cl. A61M 15/06; A61M 15/00

(52) U.S. Cl. CPC A61M 15/06; A61M 15/0021

(57) ABSTRACT

An aerosolization conduit assembly is provided for drug-delivery systems. The aerosolization conduit assembly comprises an outer tube; an inner tube disposed in the outer tube, having outer supporter openings formed on a side of wall of the inner tube; a liquid storage chamber formed between the inner tube and the outer tube; a liquid inlet opening on a side wall of the outer tube; and an atomizing member having an end arranged in the one or more outer supporter openings. The inner tube comprises parallel tubes extending from the atomizing member and feeding a double-barrel, ricocheting vortex-effect mouthpiece. The mouthpiece cooperates with the parallel tubes to allow for increased adiabatic expansion and compression, thereby providing a less intense, more even experience to a practitioner of the electronic cigarette.

### PRIORITY

This application claims the benefit of and priority to U.S. Provisional Application No. 62/867,435, filed Jun. 27, 2019, the entirety of which is incorporated herein by reference.

### FIELD

Embodiments of the present disclosure generally relate to the field of electronic drug-delivery systems. More specifically, embodiments of the disclosure relate to aerosolizing conduits for drug-delivery systems.

### BACKGROUND

Electronic cigarettes, Electronic Nicotine Delivery Systems (ENDS), and vaping devices, in general, have been shown to be safer alternatives compared to conventional burn-down tobacco products. Vaping remains a mostly physical event as compared to the chemical reactions that occur during the incomplete combustion of tobacco that occurs during conventional cigarette smoking. Outside of trace, nominal, or negligible side-products, when compared to tobacco smoking, the physical event of forming an aerosol from glycerol or another aerosol-former solution will not generate the level of toxins formed during the incomplete combustion of tobacco.

Whereas tobacco smoking remains a chemical reaction with oxygen, resulting in the incomplete combustion of tobacco, vaping is a relatively low temperature, controlled aerosolization of aerosol forming agent(s) contained in an electronic cigarette device. There are nearly 9600 chemical components found in tobacco and tobacco smoke. The number of chemicals potentially found in ENDS products is a couple of hundred at best. Each individual ENDS product generally includes orders of magnitude less chemicals than those generated by the incomplete combustion of cigarettes. As such, there is a continuing interest in developing alternative smoking products that offer less harmful ENDS consumption and that are optimized for end-user and exposed non-user safety while mitigating appeal and exposure to youth and non-users.

### SUMMARY

An aerosolization conduit assembly is provided for drug-delivery systems. The aerosolization conduit assembly comprises an outer tube; an inner tube disposed in the outer tube, having outer supporter openings formed on a side of wall of the inner tube; a liquid storage chamber formed between the inner tube and the outer tube; a liquid inlet opening on a side wall of the outer tube; and an atomizing member having an end arranged in the one or more outer supporter openings. The inner tube comprises parallel tubes extending from the atomizing member and feeding a double-barrel, ricocheting vortex-effect mouthpiece. The mouthpiece cooperates with the parallel tubes to allow for increased adiabatic expansion and compression, thereby providing a less intense, more even experience to a practitioner of the electronic cigarette.

In an exemplary embodiment, an aerosolization conduit assembly comprises: an outer capillary, cannula, and/or duct feeding a double-barrel, ricocheting vortex-effect mouthpiece; an inner capillary, cannula, and/or duct fastened and/or poised inside the outer capillary, cannula, and/or duct connecting any pored-hotplate, fractal, mesh, or coil aerosolization chamber, chimney-stack, or component; a liquid storage chamber formed between an outer surface of the inner capillary, cannula, and/or duct and an inner surface of the outer capillary, cannula, and/or duct with variations thereof; one or more bored-through and/or perforated aperture, cavity, or perforations formed on a lateral side of the outer capillary, cannula, and/or duct, communicating with the liquid storage chamber; an aerosolizing chamber formed inside the inner capillary, cannula, and/or duct comprising reinforced inserts, chimney-stack housing, and/or variable pored-hotplate, fractal, mesh, or coil aerosolizer or a combination thereof; one or more first breaches formed on a lateral side of the inner capillary, cannula, and/or duct, communicating with the liquid storage chamber; and a liquid-guiding element fastened and/or poised inside the aerosolizing chamber, wherein the liquid-guiding element is mounted on the one or more first breaches of the inner capillary, cannula, and/or duct such that ends of the liquid-guiding element are in fluid communicating with the liquid storage chamber.

In another exemplary embodiment, the one or more first breaches are formed with a notch shape comprising any one of following: a diamond shape, a U shape, an angular shape, a V shape, a half-circular shape, a half-oval shape, a half-square shape, or a half-rectangular shape. In another exemplary embodiment, the one or more first breaches are formed with a through-aperture, cavity, or perforation shape comprising any one of the following: a diamond, a circle, an eclipse, an oval, a square, or a rectangle. In another exemplary embodiment, there are at least two first breaches, and wherein at least one of the at least two first breaches is formed at a location different from a location of the remainder of the at least two first breaches on the lateral side of the inner capillary, cannula, and/or duct. In another exemplary embodiment, the aerosolization conduit assembly further comprises a vapor passage arranged at a top portion of the inner capillary, cannula, and/or duct, communicating with the aerosolizing chamber. In another exemplary embodiment, the aerosolization conduit assembly further comprises a seal member arranged at a top portion of the liquid storage chamber, sealing the top portion of the liquid storage chamber. In another exemplary embodiment, the aerosolization conduit assembly further comprises a connecting seat provided under the outer capillary, cannula, and/or duct, supporting the outer capillary, cannula, and/or duct.

In another exemplary embodiment, the aerosolization conduit assembly further comprises a holder fastened and/or poised inside the inner capillary, cannula, and/or duct. In another exemplary embodiment, the holder comprises one or more second breaches formed on a lateral side of the holder; and wherein the one or more second breaches have shapes, sizes, and are fastened and/or poised at locations on the holder corresponding to those of the first breaches on the inner capillary, cannula, and/or duct. In another exemplary embodiment, portions of the liquid-guiding element are mounted in the one or more second breaches of the holder. In another exemplary embodiment, the holder is formed of a heat retaining material including a ceramic material.

In another exemplary embodiment, the outer capillary, cannula, and/or duct feeding a double-barrel, ricocheting vortex-effect mouthpiece, an inner capillary, cannula, and/or duct fastened and/or poised inside the outer capillary, cannula, and/or duct connecting any pored-hotplate, fractal, mesh, or coil aerosolization chamber, chimney-stack, or component, a liquid storage chamber formed between an outer surface of the inner capillary, cannula, and/or duct and an inner surface of the outer capillary, cannula, and/or duct with variations thereof, one or more bored-through and/or perforated aperture, cavity, or perforations formed on a lateral side of the outer capillary, cannula, and/or duct, communicating with the liquid storage chamber, one or more first breaches formed on a lateral side of the inner capillary, cannula, and/or duct, communicating with the liquid storage chamber, a liquid-guiding element fastened and/or poised inside the aerosolizing chamber, wherein the liquid-guiding element is mounted on the one or more first breaches of the inner capillary, cannula, and/or duct such that ends of the liquid-guiding element are in fluid communicating with the liquid storage chamber, and/or variable, interchangeable combinations thereof including external and/or internal breaches, carburetor(s), exhaust-port(s), perforations and/or vent(s) leading to multiple geometries depending on and targeting for optimal aerosolization.

In an exemplary embodiment, an aerosolizer head assembly comprises: an aerosolizing capillary, cannula, and/or duct having an aerosolizing chamber; and two or more aerosolizing members for aerosolizing liquid fastened and/or poised inside the aerosolizing chamber; wherein the two more aerosolizing members are electrically connected in parallel.

In another exemplary embodiment, the two or more aerosolizing members are formed with a shape comprising any of following: a pipe, a helix, a thread, or a bar. In another exemplary embodiment, axes of the two or more aerosolizing members are spatially arranged to be perpendicular to an axis of the aerosolizing chamber. In another exemplary embodiment, axes of the two or more aerosolizing members are spatially arranged to be in parallel to an axis of the aerosolizing chamber. In another exemplary embodiment, at least one of the two or more aerosolizing members is spatially arranged to be perpendicular, parallel, skew, or orthogonal to an axis of the aerosolizing chamber, and remainder of the two or more aerosolizing members are spatially arranged to be in parallel and/or in-line to an axis of the aerosolizing chamber.

In another exemplary embodiment, an axis of at least one of the two or more aerosolizing members is inclined at an angle with respect to an axis of the aerosolizing chamber. In another exemplary embodiment, an axis of at least one of the two or more aerosolizing members is spatially arranged to be perpendicular and/or orthogonal to axes of the remainder of the two or more aerosolizing members. In another exemplary embodiment, an axis of at least one of the two or more aerosolizing members is spatially inclined at an angle with respect to axes of remainder of the two or more aerosolizing members. In another exemplary embodiment, the two or more aerosolizing members each comprises a heating element made of a ceramic, nonmetallic, metallic, and/or a combination thereof conductive material.

In another exemplary embodiment, the aerosolizer head assembly further comprises the outer capillary, cannula, and/or duct feeding a double-barrel, ricocheting vortex-effect mouthpiece, an inner capillary, cannula, and/or duct fastened and/or poised inside the outer capillary, cannula, and/or duct connecting any pored-hotplate, fractal, mesh, or coil aerosolization chamber, chimney-stack, or component, a liquid storage chamber formed between an outer surface of the inner capillary, cannula, and/or duct and an inner surface of the outer capillary, cannula, and/or duct with variations thereof, one or more bored-through and/or perforated aperture, cavity, or perforations formed on a lateral side of the outer capillary, cannula, and/or duct, communicating with the liquid storage chamber, one or more first breaches formed on a lateral side of the inner capillary, cannula, and/or duct, communicating with the liquid storage chamber, a liquid-guiding element fastened and/or poised inside the aerosolizing chamber, wherein the liquid-guiding element is mounted on the one or more first breaches of the inner capillary, cannula, and/or duct such that ends of the liquid-guiding element are in fluid communicating with the liquid storage chamber, and/or variable, interchangeable combinations thereof including external and/or internal breaches, carburetor(s), exhaust-port(s), perforations and/or vent(s) leading to multiple geometries depending on and targeting for optimal aerosolization.

### BRIEF DESCRIPTION OF THE DRAWINGS

The drawings refer to embodiments of the present disclosure in which:

FIG. 1 illustrates an exemplary embodiment of an electronic drug-delivery system, in accordance with the present disclosure;

FIG. 2 illustrates an exemplary embodiment of an electronic drug-delivery system according to the present disclosure;

FIG. 2A illustrates an exemplary embodiment of an electronic drug-delivery system, in accordance with the present disclosure;

FIG. 3 illustrates a cross-sectional view of the atomizing head assembly, in accordance with the present disclosure; and

FIG. 4 illustrates a cross-sectional view of the atomizing head assembly of FIG. 3 rotated 90° according to the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first cannula,” may be made. However, the specific numeric reference should not be interpreted as a literal sequential order but rather interpreted that the “first cannula” is different than a “second cannula.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Electronic Nicotine Delivery Systems (ENDS) are known to be safer alternatives to conventional burn-down tobacco products. Although tobacco smoking results in the production of nearly 9600 chemical components, vaping is a relatively low temperature, controlled aerosolization of aerosol forming agent(s) that produces a couple of hundred chemical components at best. Each individual ENDS product generally includes orders of magnitude less chemicals than those generated by the incomplete combustion of tobacco. As such, there is a continuing interest in developing alternative smoking products that offer less harmful ENDS consumption and that are optimized for end-user and exposed non-user safety while mitigating appeal and exposure to youth and non-users.

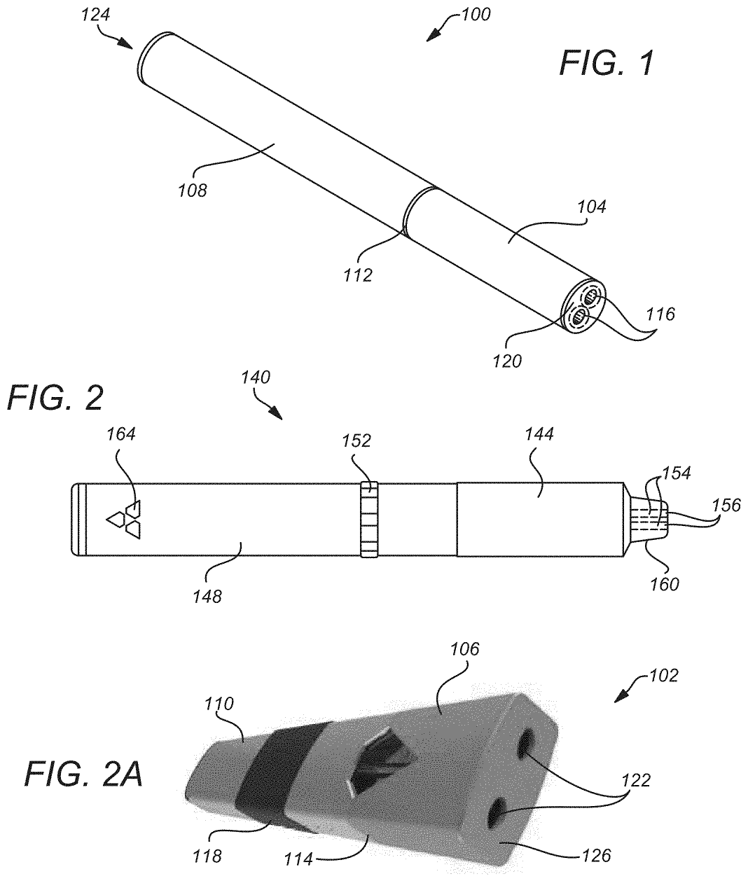

FIG. 1 illustrates an exemplary embodiment of an electronic drug-delivery system (hereinafter, “electronic cigarette”) 100, in accordance with the present disclosure. As shown in FIG. 1, the electronic cigarette 100 includes a first portion 104 and a second portion 108 that are coupled together by way of a threaded connection 112 or other convenience such as a snug-fit, detent, clamp, clasp, and/or magnets. In general, the first portion 104 comprises a replaceable cartridge, and the second portion 108 comprises a reusable fixture. The first portion 104 comprises an outer tube or casing that extends in a longitudinal direction. The second portion 108 also comprises an outer tube or casing extending in the longitudinal direction. In some embodiments, wherein the electronic cigarette 100 is disposable, the outer tube may be a single tube that houses the first and second portions 104, 108.

In general, the first portion 104 includes a pair of parallel inner tubes extending longitudinally within the first portion 104 from outlets 116 disposed in a mouthpiece portion 120 to an aerosolization chamber that includes a heater. The heater may comprise a pored-hotplate, a wire coil, a planar body, a ceramic body, a single wire, a cage of resistive wire or any other suitable form. A wick may be in communication with a liquid material contained in a liquid supply reservoir and in communication with the heater such that the wick disposes the liquid material in proximate relation to the heater. The wick preferably comprises a material having a capacity to draw the liquid material from the liquid supply reservoir by way of capillary action. A power supply disposed in the second portion 108 may be configured to apply voltage across the heater. A heater activation light 124 may be included in the second portion 108 and configured to illuminate when the heater is activated by the power supply. The first portion 104 also preferably includes at least one air inlet to deliver air to the aerosolization chamber and the pair of parallel inner tubes.

The mouthpiece portion 120 may comprise a mouth end insert having at least two off-axis outlets 116. The mouthpiece portion 120 preferably is of a ricocheting vortex-effect variety configured to cooperate with the abovementioned pair of parallel inner tubes to optimize the pressure for various formulations to properly distribute through the microfluidic cell by capillary effect triggered by the adiabatic manipulation the mouthpiece and conduit offer per formulation viscosity. It is contemplated that the combination of the mouthpiece portion 120 and the parallel inner tubes allow for increased adiabatic expansion and compression, thereby providing a less intense, more even experience to a practitioner of the electronic cigarette 100. It is contemplated that the mouthpiece portion 120 and parallel inner tubes operate to reduce the risk of harm to exposed tissues by minimizing addiction potential of induced tactile responses instead of maximizing those and downstream dependence mechanisms.

FIG. 2 illustrates an exemplary embodiment of an electronic cigarette 140 according to the present disclosure. The electronic cigarette 140 is substantially similar to the electronic cigarette 100 shown in FIG. 1, with the exception that the electronic cigarette 140 is includes a size that resembles a cigar. As shown in FIG. 2, the electronic cigarette 140 comprises a first portion 144 and a second portion 148 that are joined by way of a connection 152. The connection 152 may comprises a threaded connection or other convenience such as a snug-fit, detent, clamp, clasp, and/or magnets. The first portion 144 may comprise a replaceable cartridge, and the second portion 148 may comprise a reusable fixture. The first portion 144 generally comprises an outer tube or casing that extends in a longitudinal direction, and the second portion 148 also comprises an outer tube or casing that extends in the longitudinal direction. As described herein, in some embodiments, wherein the electronic cigarette 140 is disposable, the outer tube may be a single tube that houses the first and second portions 144, 148.

As shown in FIG. 2, the first portion 144 includes a pair of parallel inner tubes 154 extending longitudinally within the first portion 144 from outlets 156 disposed in a mouthpiece portion 160 to an aerosolization chamber that includes a heater. It is contemplated that the heater may comprise any of a pored-hotplate, a wire coil, a planar body, a ceramic body, a single wire, a cage of resistive wire or any other suitable form. A wick may be in communication with a liquid contained in a liquid supply reservoir and in communication with the heater such that the wick disposes the liquid in proximate relation to the heater. The wick preferably comprises a material capable of drawing the liquid from the liquid supply reservoir by way of capillary action. A power supply disposed in the second portion 148 may be configured to apply voltage across the heater. A heater activation light 164 may be included in the second portion 148 and configured to illuminate when the heater is activated by the power supply. The first portion 144 also preferably includes at least one air inlet to deliver air to the aerosolization chamber and the pair of parallel inner tubes.

The mouthpiece portion 160 may include at least two outlets 156. The mouthpiece portion 160 preferably is of a ricocheting vortex-effect variety configured to cooperate with the abovementioned pair of parallel inner tubes to optimize the pressure for various formulations to properly distribute through the microfluidic cell by capillary effect triggered by the adiabatic manipulation the mouthpiece and conduit offer per formulation viscosity. It is envisioned that the combination of the mouthpiece portion 160 and the parallel inner tubes allow for increased adiabatic expansion and compression, thereby providing a less intense, more even experience to a practitioner of the electronic cigarette 140. It is contemplated that the mouthpiece portion 160 and parallel inner tubes operate to reduce the risk of harm to exposed tissues by minimizing addiction potential of induced tactile responses instead of maximizing those and downstream dependence mechanisms.

FIG. 2A illustrates an exemplary embodiment of an electronic drug-delivery system 102, in accordance with the present disclosure. The electronic cigarette 102 includes a first portion 106 and a second portion 110 that are coupled together by way of a connection 114. The connection 114 may be achieved by way of a snug-fit, detent, clamp, clasp, magnets, or other suitable convenience. In general, the first portion 106 comprises a replaceable cartridge, and the second portion 110 comprises a reusable fixture. The first portion 106 comprises an outer casing that extends in a longitudinal direction. The second portion 110 also comprises an outer casing extending in the longitudinal direction. In some embodiments, wherein the electronic cigarette 102 is disposable, the outer casing may be a single casing that houses the first and second portions 106, 110. Further, as shown in FIG. 2A, a comfort sleeve 118 may be disposed on the second portion 110 for ergonomic purposes. The comfort sleeve 118 may be comprised of an of silicone, Latex, rubber, or other pliable material, without limitation.

In general, the first portion 106 includes a pair of parallel inner tubes extending longitudinally within the first portion 106 from outlets 122 disposed in a mouthpiece portion 126 to an aerosolization chamber that includes a heater. The heater may comprise a wire coil, a planar body, a ceramic body, a single wire, a cage of resistive wire or any other suitable form. A wick may be in communication with a liquid material contained in a liquid supply reservoir and in communication with the heater such that the wick disposes the liquid material in proximate relation to the heater. The wick preferably comprises a material having a capacity to draw the liquid material from the liquid supply reservoir by way of capillary action. A power supply disposed in the second portion 110 may be configured to apply voltage across the heater. A heater activation light 164 (see FIG. 2) may be included in the second portion 110 and configured to illuminate when the heater is activated by the power supply. Further, in some embodiments, the heater activation light 164 may include a tricolor LED (light emitting diode) and be configured to indicate cell capacity of the power supply. The first portion 106 also preferably includes at least one air inlet to deliver air to the aerosolization chamber and the pair of parallel inner tubes.

The mouthpiece portion 126 may comprise a mouth end cap having at least two off-axis outlets 122. The mouthpiece portion 126 preferably is of a ricocheting vortex-effect variety configured to cooperate with the abovementioned pair of parallel inner tubes to optimize the pressure for various formulations to properly distribute through the microfluidic cell by capillary effect triggered by the adiabatic manipulation the mouthpiece and conduit offer per formulation viscosity. It is contemplated that the combination of the mouthpiece portion 126 and the parallel inner tubes allow for increased adiabatic expansion and compression, thereby providing a less intense, more even experience to a practitioner of the electronic cigarette 102. It is contemplated that the mouthpiece portion 126 and parallel inner tubes operate to reduce the risk of harm to exposed tissues by minimizing addiction potential of induced tactile responses instead of maximizing those and downstream dependence mechanisms.

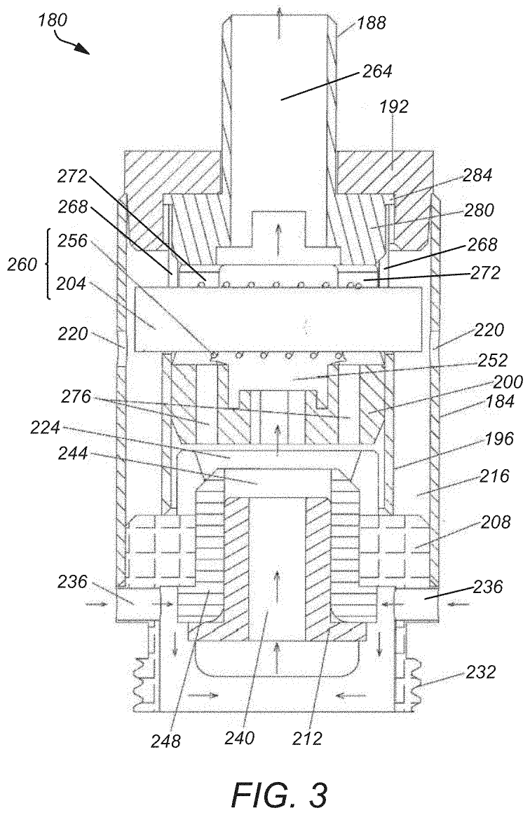

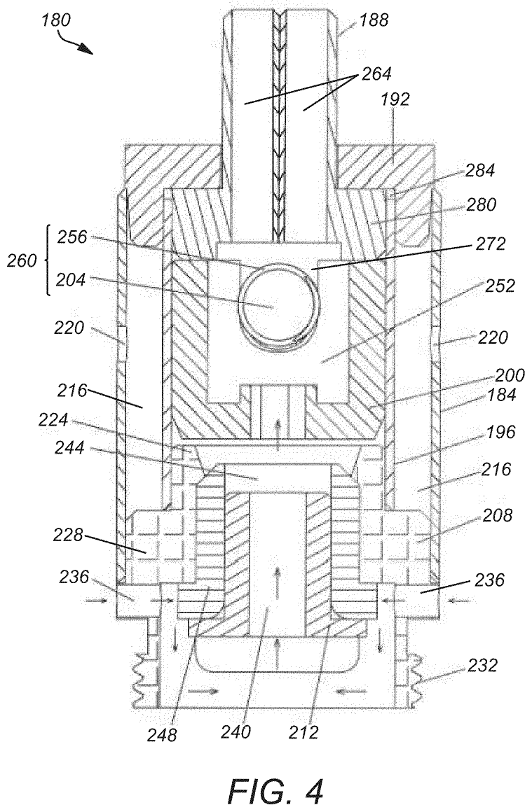

Turning now to FIGS. 3-4, an exemplary embodiment of an atomizer head assembly 180 that may be incorporated into the electronic cigarettes 100, 140 of FIGS. 1-2 is shown, according to the present disclosure. FIG. 3 illustrates a cross-sectional view of the atomizer head assembly 180 while FIG. 4 illustrates a cross-sectional view of the atomizer head assembly 180 of FIG. 3 rotated 90°.

As shown in FIGS. 3-4, the atomizer head assembly 180 includes an outer tube 184, a vapor passage 188, a seal member 192, an inner tube 196, a support unit 200, one or more atomizing members 204, a connecting seat 208, and a conductive member 212. The atomizer head assembly 180 may be disposed inside an atomizer head shell (not shown) that also contains a liquid storage reservoir that stores a liquid to be atomized by the atomizer head assembly 180.

The outer tube 184 comprises a hollow tube body that is mounted on the connecting seat 208 of the atomizer head assembly 180. The inner tube 196 generally is centrally disposed within the outer tube 184 such that a liquid storage chamber 216 is formed by a wall of the inner tube 196 and a wall of outer tube 184 to store liquid. One or more liquid inlet openings 220 may be provided on a lateral side of the wall of outer tube 184 and may be shaped, sized, and disposed at a location of wall of the outer tube 184 to allow the liquid stored in an atomizer shell (not show) to flow through the liquid inlet openings 220 into the liquid storage chamber 216. The liquid inlet openings 220 can be through holes of any geometric shape including, but not necessarily limited to, circles, ecliptics, ovals, squares, rectangles, or any combination thereof.

The connecting seat 208 on which the outer tube 184 is mounted includes a seat neck 224, a seat body 228, and a thread 232. The seat neck 224 has an outer diameter substantially the same as the inner diameter of inner tube 196 such that the inner tube 196 is press fit onto the seat neck 224 to be fixed with the connecting seat 208. Further, the thread 232 is coupled to the atomizer head shell (not shown) to connect the atomizer head assembly 180 with the atomizer head shell.

Moreover, an air intake hole 236 is provided on a lateral wall of the connecting seat 208 through which air is taken from the atomizer head shell (not shown) into a communicating passage 240 centrally formed inside the conductive member 212 and subsequently, flows through an air passage 244 centrally formed inside an insulating part 248, air channels inside the inner tube 196 and the support unit 200, and flows into an atomizing chamber 252 formed inside the inner tube 196 and the support unit 200.

The insulating part 248 is centrally arranged inside the connecting seat 208 by press fitting into the connecting seat 208 to electrically insulate the connecting seat 208 from currents generated by the conductive member 212. Further, the conductive member 212 is centrally arranged inside the insulating part 248 by press fitting into the insulating part 248.

The conductive member 212 operates as a conductive positive electrode with one end connected with a first lead wire (not shown) of heating elements 256 and with the other end connected with a power source (not shown), such as a battery, external to the atomizer head assembly. The connecting seat 208 operates as conductive negative electrode with one end connected with a second lead wire (not shown) of heating elements 256 comprising atomizing members 260 and with the other end connected with a power source (not shown), such as a battery, external to the atomizer head assembly 180. Thus, current generated by the power source is provided to the heating elements 256 through the conductive member 212, the connecting seat 208, and the lead wires of heating elements 256.

As shown in FIGS. 3-4, the inner tube 196 arranged inside the outer tube 184 is formed with a hollow tube body and is mounted on the connecting seat 208 by press fitting with the seat neck 224 of the connecting seat 208. As discussed above, with the inner tube 196 is centrally arranged within the outer tube 184, the liquid storage chamber 216 is formed by a wall of the inner tube 196 and a wall of the outer tube 184 to store the liquid.

The atomizing chamber 252 is formed with a cylindrical shape inside the inner tube 196. During operating, liquid contained in the atomizing member 260 is heated in the atomizing chamber 252 to a sufficiently high temperature by the heating elements 256 to be atomized into aerosols or fine droplets. Air from the bottom of the atomizer head assembly 180 flows over the liquid guide element 204 to entrain the aerosols or fine droplets of the liquid and flow upward through a pair of parallel tubes 264 within the vapor passage 188 to exit the atomizer head assembly 180.

As best shown in FIG. 3, the inner tube 196 includes one or more outer supporter openings 268 provided on a lateral wall of the inner tube 196. The outer supporter openings 268 may be formed as notches with predetermined depths that are vertically measured from top edges of the notches to the bottom of the notches. The outer supporter openings 268 in forms of notches may take any geometric shape including, but not necessarily limited to, U shapes, angular shapes, V shapes, half-circular shapes, half-oval shapes, half-square shapes, half-rectangular shapes, or any combination thereof. As such, the atomizing member 260 can be securely supported on the wall of inner tube 196 by mounting ends of the atomizing member 260 in the notches.

In some embodiments, the outer supporter openings 268 may comprise through holes on the lateral side of the wall of inner tube 196 with predetermined depths that are vertically measured from a top edge of the wall of inner tube 196 to the bottom of the through holes. The outer supporter openings 268 in the form of through holes may include any geometric shape such as, but not limited to, circles, ecliptics, ovals, squares, rectangles, or any combination thereof. As a result, the atomizing member 260 can be securely supported by the wall of inner tube 196 by inserting ends of the atomizing member 260 through the through holes.

Moreover, it is contemplated that, in some embodiments, the inner tube 196 may comprise a plurality of outer supporter openings 268 in a notch shape or a through-hole shape where the plurality of outer supporter openings 268 is formed at different depths, allowing a plurality of atomizing members 260 to be mounted in the outer supporter openings 268 with various spatial configurations. Orientations of the atomizing members 260 within the plurality of outer supporter openings 268 will be apparent to those skilled in the art.

In some embodiments, a support unit 200 may be optionally included in the inner tube 196. When the support unit 200 is included in the inner tube 196, the support unit 200 may be press fit into the inner tube 196 since the support unit 200 has an outer diameter substantially the same as the inner diameter of inner tube 196. The support unit 200 may be formed as a hollow tube body with one or more inner supporter openings 272 disposed on a lateral wall of the support unit 200 for providing a supporting base for the atomizing member 260. As such, the atomizing member 260 may be mounted in both the inner supporter openings 272 of support unit 200 and the outer supporter openings 268 of inner tube 196.

The inner supporter openings 272 may be shaped, sized, and deposited at locations corresponding to the outer supporter openings 268 of the inner tube 196 to allow the atomizing member 260 penetrate both the outer supporter openings 268 and the inner supporter openings 272. In an exemplary embodiment, the inner supporter openings 272 and the outer supporter openings 268 are aligned such that centers of the inner supporter openings 272 and the outer supporter openings 268 are substantially coincide.

In some embodiments, the inner supporter openings 272 may be formed as notches having predetermined depths that are vertically measured from top edges of the notches to the bottom of the notches. The inner supporter openings 272 in the form of notches may take any geometric shape including, but not necessarily limited to, U shapes, angular shapes, V Shapes, half-circular shapes, half-oval shapes, half-square shapes, half-rectangular shapes, or any combination thereof. As such, the atomizing member 260 may be securely supported on the wall of the support unit 200 by mounting ends of the atomizing member 260 in the notches.

In some embodiments, the inner supporter openings 272 may be formed as through holes on the side of the wall of support unit 200 with predetermined depths that are vertically measured from a top edge of the wall of support unit 200 to the bottom of the through holes. The inner supporter openings 272 in the form of through holes may take any geometric shape including, but not necessarily limited to, circles, ecliptics, ovals, squares, rectangles, or any combination thereof. As such, the atomizing member 260 may be securely supported in the wall of the support unit 200 by inserting ends of the atomizing member 260 through the through holes.

Moreover, in some embodiments, the support unit 200 may include two or more through holes 276 vertically pierced through a wall of the support unit 200 such that two lead wires (not shown) of the heating element 256 penetrating through these through holes 276 to connect the heating element 256 with the conductive member 212 and the connecting seat 208, respectively. Thus, electricity may be provided from a power source (not shown), such as a battery, external to the atomizer head assembly 180 to the heating element 256 through the conductive member 212, the connecting seat 208, and the lead wires.

In some embodiments, the liquid guide element 204 may be made of a porous material, including but not necessarily limited to, a fibrous material, a ceramic material, or a combination of both. When liquid passing through the liquid inlet opening 220 of the outer tube 184 enters into the liquid storage chamber 216 formed between the inner tube 196 and the outer tube 184 and the level of the liquid in the liquid storage chamber 216 is sufficiently high, the liquid contacts the liquid guide element 204 and the liquid guide element 204 takes in some of the liquid. The liquid thereby subsequently permeates the liquid guide element 204.

The heating element 256 is coupled with the liquid guide element 204 for heating and atomizing the liquid taken in by the liquid guide element 204. The liquid guide element 204 and the heating element 256 may be coupled in any of various configurations. The heating element 256 may be coupled with a lead wire (not shown) through which the heating element 256 may be connected with a power source (not shown), such as a battery. As such, electricity may be supplied to the heating element 256 by the power source such that the heating element 256 generates heat to atomize the liquid in the liquid guide element 204.

With continuing reference to FIGS. 3-4, when the heating element 256 heats the liquid absorbed in the liquid guide element 204 to a sufficiently high temperature in the atomizing chamber 252, the liquid is atomized into aerosols or fine droplets. Air entering from the air intake hole 236 of the connecting seat 208 and flowing upward to the atomizing chamber 252 entrains the aerosols or fine droplets to flow up through a pair of parallel tubes 264 inside the vapor passage 188.

The vapor passage 188 may be centrally disposed inside the inner tube 196 and include a base portion 280 at the bottom, the parallel tubes 264, and a sealing disc 284 connecting the base portion 280 and the parallel tubes 264. The hollow interiors of the base portion 280, the parallel tubes 264, and the sealing disc 284 integrally form the vapor passage 188 such that the atomized liquid generated inside the atomizing chamber 252 is entrained by the up-flowing air to flow upward and exit the atomizer head assembly 180 through the vapor passage 188.

As shown in FIGS. 3-4, the liquid storage chamber 216 formed between the inner tube 196 and the outer tube 184 is sealed from the top by the seal member 192 to prevent liquid in the liquid storage chamber 216 from leaking out of the atomizer head assembly 180. The seal member 192 has a circular ring structure with an opening in the center. The vapor passage 188 extends through the central opening of the seal member 192. The seal member 192 may be press fit between the inner tube 196 and the outer tube 184 to seal the liquid storage chamber 216. Moreover, the seal member 192 may have an outer diameter substantially the same as the inner diameter of outer tube 184 and an inner diameter substantially the same as the outer diameter of inner tube 196. The seal member 192 may be made with any of various elastic materials including, but not necessarily limited to, silica gel, rubber, plastic, or elastic alloy, or any combination thereof.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. An aerosolization conduit assembly, comprising:
an outer capillary, cannula, and/or duct feeding a double-barrel, ricocheting vortex-effect mouthpiece;
an inner capillary, cannula, and/or duct fastened and/or poised inside the outer capillary, cannula, and/or duct connecting any pored-hotplate, fractal, mesh, or coil aerosolization chamber, chimney-stack, or component;
a liquid storage chamber formed between an outer surface of the inner capillary, cannula, and/or duct and an inner surface of the outer capillary, cannula, and/or duct with variations thereof;
one or more bored-through and/or perforated aperture, cavity, or perforations formed on a lateral side of the outer capillary, cannula, and/or duct, communicating with the liquid storage chamber;
an aerosolizing chamber formed inside the inner capillary, cannula, and/or duct comprising reinforced inserts, chimney-stack housing, and/or variable pored-hotplate, fractal, mesh, or coil aerosolizer or a combination thereof;
one or more first breaches formed on a lateral side of the inner capillary, cannula, and/or duct, communicating with the liquid storage chamber; and
a liquid-guiding element fastened and/or poised inside the aerosolizing chamber, wherein the liquid-guiding element is mounted on the one or more first breaches of the inner capillary, cannula, and/or duct such that ends of the liquid-guiding element are in fluid communicating with the liquid storage chamber.

2. The aerosolization conduit assembly of claim 1, wherein the one or more first breaches are formed with a notch shape comprising any one of the following: a diamond shape, a U shape, an angular shape, a V shape, a half-circular shape, a half-oval shape, a half-square shape, or a half-rectangular shape.

3. The aerosolization conduit assembly of claim 1, wherein the one or more first breaches are formed with a through-aperture, cavity, or perforation shape comprising any one of following: a diamond, a circle, an eclipse, an oval, a square, or a rectangle.

4. The aerosolization conduit assembly of claim 1, wherein there are at least two first breaches, and wherein at least one of the at least two first breaches is formed at a location different from a location of the remainder of the at least two first breaches on the lateral side of the inner capillary, cannula, and/or duct.

5. The aerosolization conduit assembly of claim 1, further comprising a vapor passage arranged at a top portion of the inner capillary, cannula, and/or duct, communicating with the aerosolizing chamber.

6. The aerosolization conduit assembly of claim 1, further comprising a seal member arranged at a top portion of the liquid storage chamber, sealing the top portion of the liquid storage chamber.

7. The aerosolization conduit assembly of claim 1, further comprising a connecting seat provided under the outer capillary, cannula, and/or duct, supporting the outer capillary, cannula, and/or duct.

8. The aerosolization conduit assembly of claim 1, further comprising a holder fastened and/or poised inside the inner capillary, cannula, and/or duct.

9. The aerosolization conduit assembly of claim 8, wherein the holder comprises one or more second breaches formed on a lateral side of the holder; and wherein the one or more second breaches have shapes, sizes, and are fastened and/or poised at locations on the holder corresponding to those of the first breaches on the inner capillary, cannula, and/or duct.

10. The aerosolization conduit assembly of claim 9, wherein portions of the liquid-guiding element are mounted in the one or more second breaches of the holder.

11. The aerosolization conduit assembly of claim 8, wherein the holder is formed of a heat retaining material including a ceramic material.

12. The aerosolization conduit assembly of claim 1, wherein the outer capillary, cannula, and/or duct feeding a double-barrel, ricocheting vortex-effect mouthpiece, an inner capillary, cannula, and/or duct fastened and/or poised inside the outer capillary, cannula, and/or duct connecting any pored-hotplate, fractal, mesh, or coil aerosolization chamber, chimney-stack, or component, a liquid storage chamber formed between an outer surface of the inner capillary, cannula, and/or duct and an inner surface of the outer capillary, cannula, and/or duct with variations thereof, one or more bored-through and/or perforated aperture, cavity, or perforations formed on a lateral side of the outer capillary, cannula, and/or duct, communicating with the liquid storage chamber, one or more first breaches formed on a lateral side of the inner capillary, cannula, and/or duct, communicating with the liquid storage chamber, a liquid-guiding element fastened and/or poised inside the aerosolizing chamber, wherein the liquid-guiding element is mounted on the one or more first breaches of the inner capillary, cannula, and/or duct such that ends of the liquid-guiding element are in fluid communicating with the liquid storage chamber, and/or variable, interchangeable combinations thereof including external and/or internal breaches, carburetor(s), exhaust-port(s), perforations and/or vent(s) leading to multiple geometries depending on and targeting for optimal aerosolization.

13. An aerosolizer head assembly, comprising:
an aerosolizing capillary, cannula, and/or duct having an aerosolizing chamber; and
two or more aerosolizing members for aerosolizing liquid fastened and/or poised inside the aerosolizing chamber;
wherein the two more aerosolizing members are electrically connected in parallel.

14. The aerosolizer head assembly of claim 13, wherein the two or more aerosolizing members are formed with a shape comprising any of following: a pipe, a helix, a thread, or a bar.

15. The aerosolizer head assembly of claim 13, wherein axes of the two or more aerosolizing members are spatially arranged to be perpendicular to an axis of the aerosolizing chamber.

16. The aerosolizer head assembly of claim 13, wherein axes of the two or more aerosolizing members are spatially arranged to be in parallel to an axis of the aerosolizing chamber.

17. The aerosolizer head assembly of claim 13, wherein at least one of the two or more aerosolizing members is spatially arranged to be perpendicular, parallel, skew, or orthogonal to an axis of the aerosolizing chamber, and remainder of the two or more aerosolizing members are spatially arranged to be in parallel and/or in-line to an axis of the aerosolizing chamber.

18. The aerosolizer head assembly of claim 13, wherein an axis of at least one of the two or more aerosolizing members is inclined at an angle with respect to an axis of the aerosolizing chamber.

19. The aerosolizer head assembly of claim 15, wherein an axis of at least one of the two or more aerosolizing members is spatially arranged to be perpendicular and/or orthogonal to axes of the remainder of the two or more aerosolizing members.

20. The aerosolizer head assembly of claim 15, wherein an axis of at least one of the two or more aerosolizing members is spatially inclined at an angle with respect to axes of remainder of the two or more aerosolizing members.

21. The aerosolizer head assembly of claim 13, wherein the two or more aerosolizing members each comprises a heating element made of a ceramic, nonmetallic, metallic, and/or a combination thereof conductive material.

22. The aerosolizer head assembly of claim 13, wherein the outer capillary, cannula, and/or duct feeding a double-barrel, ricocheting vortex-effect mouthpiece, an inner capillary, cannula, and/or duct fastened and/or poised inside the outer capillary, cannula, and/or duct connecting any pored-hotplate, fractal, mesh, or coil aerosolization chamber, chimney-stack, or component, a liquid storage chamber formed between an outer surface of the inner capillary, cannula, and/or duct and an inner surface of the outer capillary, cannula, and/or duct with variations thereof, one or more bored-through and/or perforated aperture, cavity, or perforations formed on a lateral side of the outer capillary, cannula, and/or duct, communicating with the liquid storage chamber, one or more first breaches formed on a lateral side of the inner capillary, cannula, and/or duct, communicating with the liquid storage chamber, a liquid-guiding element fastened and/or poised inside the aerosolizing chamber, wherein the liquid-guiding element is mounted on the one or more first breaches of the inner capillary, cannula, and/or duct such that ends of the liquid-guiding element are in fluid communicating with the liquid storage chamber, and/or variable, interchangeable combinations thereof including external and/or internal breaches, carburetor(s), exhaust-port(s), perforations and/or vent(s) leading to multiple geometries depending on and targeting for optimal aerosolization.

23. The aerosolizer head assembly of claim 13, wherein the aerosolizer head or nebulizer conduit in its simplest iteration utilizes a double-barrel, ricocheting vortex-effect mouthpiece for optimal aerosol airflow as outlined in FIG. 2. This feature of the conduit ensures optimal airflow and maximizes adiabatic manipulation by the user independent of the modulator device's adjustable carburetor system, but combines for maximum effect compared to other power sources.
